(19) United States
(12) Patent Application Publication
(10) Pub. No.: US 2011/0124730 A1
(43) Pub. Date: May 26, 2011

(54) ORAL PHARMACEUTICAL SUSPENSION COMPRISING PARACETAMOL AND IBUPROFEN

(75) Inventors: Hartley Atkinson, Auckland (NZ); Austin Kiely, Waterford (IE)

(73) Assignee: WOCKHARDT RESEARCH CENTRE, Aurangabad, Maharashtra (IN)

(21) Appl. No.: 12/811,187

(22) PCT Filed: Jan. 3, 2008
(86) PCT No.: PCT/IB2008/000005

§ 371 Date: Jul. 8, 2010

(51) Int. Cl. A61K 31/192; A61P 29/00

(52) U.S. Cl. 514/570

(57) ABSTRACT

The present invention relates to an oral pharmaceutical suspension comprising paracetamol and ibuprofen. The invention also relates to a method of treating perioperative or postoperative pain by administering to a subject a therapeutically effective amount of oral pharmaceutical suspension comprising paracetamol and Ibuprofen.

### FIELD OF THE INVENTION

The present invention relates to an oral pharmaceutical suspension comprising paracetamol and ibuprofen wherein the said suspension is used for the treatment of preoperative, perioperative or postoperative pain.

### BACKGROUND OF THE INVENTION

Paracetamol or Acetaminophen or 4′-hydroxyacetanilide, is a non-opiate, non-salicylate analgesic and antipyretic drug. It is a peripherally acting analgesic and is well absorbed orally. It produces analgesia by elevation of the pain threshold and antipyresis through action on the hypothalamic heat-regulating center. Acetaminophen is chemically N-(4-Hydroxyphenyl)acetamide represented by Formula I. It provides temporary relief of minor aches and pains with heartburn or acid indigestion and upset stomach associated with these symptoms.

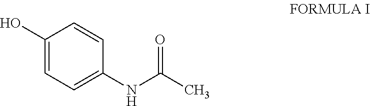

Ibuprofen, a nonsteroidal anti-inflammatory drug, possesses analgesic and antipyretic activities. Its mode of action is related to prostaglandin synthetase inhibition. Ibuprofen is chemically (±)-2-(p-isobutylphenyl) propionic acid represented by Formula II. It is indicated in the treatment for relief of the signs and symptoms of rheumatoid arthritis and osteoarthritis, mild to moderate pain and treatment of primary dysmenorrhea.

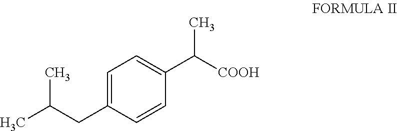

The suspension dosage form of paracetamol and ibuprofen are commercially marketed under the trade name of Ibugesic Plus® (Ibuprofen 100 mg and Paracetamol 162.5 mg), Lotem® (Ibuprofen 100 mg and Paracetamol 125 mg) and Anaflam® (Ibuprofen 100 mg and Paracetamol 125 mg).

European Application No. EP0109281 describes pharmaceutical composition of flubriprofen or ibuprofen and acetaminophen.

International (PCT) Publication WO2006004449 describes pharmaceutical composition containing Ibuprofen and Paracetamol for the treatment of pain.

Swallow J et. al. Journal of child health care: for professionals working with children in the hospital and community (2000), 4(3): 93-8 report the discharge prescription of Paracetamol and Ibuprofen to all children undergoing tonsillectomy.

Homer et. al. The Journal of laryngology and otology (2001), 115(3): 205-8 report that the Paracetamol and Ibuprofen is an effective analgesic combination in children (without asthma) following tonsillectomy.

Pickering et. al. British Journal of Anaesthesia (2002), 88(1): 72-77 report that a perioperative combination of ibuprofen and Paracetamol as a strategy in children undergoing tonsillectomy.

Hyllested, M et. al British Journal of Anaesthesia (2002), 88(2): 199-214 report that the addition of an NSAID to paracetamol may confer additional analgesic efficacy compared with paracetamol alone, and also suggest that paracetamol may enhance analgesia when added to an NSAID, compared with NSAIDs alone.

Kokki Hannu Paediatric drugs (2003), 5(2): 103-23 report that the combination of Paracetamol and Ibuprofen to improve analgesia in children undergoing tonsillectomy.

Menhinick K A et. al. International endodontic journal (2004), 37(8): 531-41 report that the combination of ibuprofen with acetaminophen may be more effective than ibuprofen alone for the management of postoperative endodontic pain.

Gazal Giath et. al. International journal of paediatric dentistry/the British Paedodontic Society [and] the International Association of Dentistry for Children (2007), 17(3): 169-77 reports evidence to support the oral administration of ibuprofen alone or in combination with paracetamol for postoperative analgesia in children who are having teeth extracted under GA.

Several other non-patent literature references report the use of paracetamol and ibuprofen combination in treatment of pain.

### SUMMARY OF THE INVENTION

One of the aspects of the present invention provides an oral pharmaceutical suspension comprising 100-500 mg/5 ml of paracetamol, 40-80 mg/5 ml of ibuprofen and one or more pharmaceutically acceptable excipients.

Another aspect of the present invention provides an oral pharmaceutical suspension comprising 200-450 mg/5 ml of paracetamol, 100-200 mg/5 ml of ibuprofen and one or more pharmaceutically acceptable excipients.

The pharmaceutical suspension of the present invention may include paracetamol or salts or derivatives thereof and ibuprofen or salts or derivatives thereof as active ingredients.

Embodiments of the pharmaceutical suspension may include one or more of the following features. For example, the pharmaceutical suspension may include one or more pharmaceutically acceptable excipients. The pharmaceutically acceptable excipients may include one or more of suspending or viscosity increasing agents, sweeteners, buffering agent, preservatives, wetting agents, flavoring agent, solvents and the like.

Another aspect of the present invention provides a method of treating preoperative, perioperative or postoperative pain by administering to a subject a therapeutically effective amount of oral pharmaceutical suspension comprising 100-500 mg/5 ml of paracetamol and 40-80 mg/5 ml of ibuprofen.

Another aspect of the present invention provides a method of treating preoperative, perioperative or postoperative pain by administering to a subject a therapeutically effective amount of oral pharmaceutical suspension comprising 200-450 mg/5 ml of paracetamol and 100-200 mg/5 ml of ibuprofen.

The phrase ‘subject’ as used herein refers to mammal.

Embodiments of the method of treating preoperative, perioperative or postoperative pain may include one or more of the following features. For example, the preoperative, perioperative or postoperative pain may be associated with one or more surgeries. The surgeries may include one or more of throat (like tonsillectomy, adenoidectomy), dental (like periodontal), ear (like myringotomy), nose and the like.

The details of one or more embodiments of the inventions are set forth in the description below. Other features, objects and advantages of the inventions will be apparent from the description and claims.

### DETAILED DESCRIPTION OF THE INVENTION

It is also known that the appropriate, effective preoperative, perioperative and postoperative analgesia are necessary to control the pain. Inadequately controlled pain results in an unwillingness or refusal to eat and drink; this can hinder recovery and early discharge. Poor pain management after discharge continues to impair the patient's ability to eat and drink adequately with the accompanying risk of dehydration, infection and secondary hemorrhage.

Use of NSAIDS in controlling the pain is well known in the art. The use of Non-steroidal anti-inflammatory drugs (NSAIDS) like ibuprofen is associated with number of side effects. The most common side effects from ibuprofen are rash, ringing in the ears, headaches, dizziness, drowsiness, abdominal pain, nausea, diarrhea, constipation and heartburn. It has been reported that the NSAIDs reduce the ability of blood to clot and therefore increase bleeding after an injury. Ibuprofen may cause ulceration of the stomach or intestine, and the ulcers may bleed. It is also reported that the NSAIDs reduce the flow of blood to the kidneys and impair function of the kidneys and individuals with asthma are more likely to experience allergic reactions to ibuprofen and other NSAIDs. Fluid retention (edema), blood clots, heart attacks, hypertension and heart failure have also been associated with the use of NSAIDs.

The present inventors while working on the paracetamol and ibuprofen suspension formulation have noticed that when a lower dose range of Ibuprofen i.e. between 40-80 mg/5 ml is combined with 100-500 mg/5 ml of paracetamol, it provides better management of preoperative, perioperative as well as postoperative pain and reduced side effects of ibuprofen (NSAIDS) as compared to the use of ibuprofen (100 mg/5 ml or more) alone. The present inventors have also noticed that the oral suspension formulation comprising 100-200 mg/5 ml of ibuprofen and 200-450 mg/5 ml of paracetamol can be used in the treatment and management of preoperative, perioperative as well as postoperative pain associated with surgeries.

The present inventors have further noticed that the suspension formulation of the present invention provides significantly better pain management following surgery, relieves discomfort that may be due to oedema, inflammation or muscle spasm, early recovery and discharge, overcome the problem of managing these two drugs separately and to improve the quality of analgesia in perioperative, postoperative and other settings.

The pharmaceutical oral suspension composition of the present invention comprises Paracetamol and ibuprofen as active ingredients. The composition of the present invention can be prepared by adding paracetamol, ibuprofen and pharmaceutically acceptable excipients to purified water followed by mixing. The pH of the obtained suspension can be adjusted in the range of 2-6 by using suitable pharmaceutically acceptable excipients followed by adding a suitable flavoring agent.

The pharmaceutically acceptable excipients may include one or more of suspending or viscosity increasing agents, sweeteners, buffering agent, preservatives, wetting agents, flavoring agent, solvents and the like.

Suitable suspending or viscosity increasing agents may include one or more of xanthan gum, guar gum, tragacanth, acacia, gelatin, carrageenan, agar-agar, povidone, alginic acid, sodium alginate, propylene glycol alginate, carbomer, magnesium aluminium silicate, carboxymethylcellulose calcium, sodium carboxymethylcellulose, ethylcellulose, methylcellulose, hydroxypropyl methylcellulose, hydroxyethylcellulose, hydroxypropylcellulose, microcrystalline cellulose, polydextrose, sucrose, sorbitol, xylitol, dextrose, fructose, maltitol, bentonite, polyvinyl alcohol, colloidal silicon dioxide, and the like.

Suitable sweeteners may include one or more of sucrose, sorbitol, xylitol, dextrose, fructose, maltitol, acesulfame potassium, aspartame, saccharin, saccharin sodium, liquid maltitol, liquid glucose, cyclamate, sodium cyclamate and the like.

Suitable buffering agents may include one or more of citric acid, sodium citrate, sodium phosphate, potassium citrate, and the like.

Suitable preservatives may include one or more of sodium benzoate, benzoic acid, ethylenediaminetetraacetic acid, sorbic acid, bronopol, butyl paraben, methyl paraben, ethylparaben, propyl paraben, sodium propionate, chlorhexidine, potassium sorbate, propylene glycol, sodium bisulfite, sodium metabisulfite, sodium salts of hydroxybenzoate and the like.

Suitable wetting agents may include one or more of polyethylene glycol, polysorbates, sorbitan esters and the like.

Suitable flavoring agents may include one or more of artificial strawberry flavor, artificial cream flavor, vanilla, cherry, raspberry and the like.

Suitable solvents may include one or more of water, glycerol, propylene glycol, polyethylene glycol, ethanol and the like.

The present invention is further illustrated by the following examples which are provided merely to be exemplary of the invention and do not limit the scope of the invention. Certain modifications and equivalents will be apparent to those skilled in the art and are intended to be included within the scope of the present invention.

### Example 1 and 2

Table 1 provides composition of batches of the present invention.

TABLE 1

| | | Example 1 | Example 2 |
|---|---|---|---|
| SN | Ingredients | mg/5 ml | mg/5 ml |
| 1. | Paracetamol | 200 | 100 |
| 2. | Ibuprofen | 100 | 40 |
| 3. | Magnesium aluminum silicate | 5-150 | 5-150 |
| 4. | Xanthan gum | 0.5-50 | 0.5-50 |
| 5. | Glycerol | 5-250 | 5-250 |
| 6. | Liquid maltitol | 1000-6000 | 1000-6000 |
| 7. | Sodium benzoate | 1-25 | 1-25 |
| 8. | Citric acid | 5-100 | 5-100 |
| 9. | Saccharin sodium | 1-30 | 1-30 |
| 10. | Polysorbate 80 | 1-50 | 1-50 |
| 11. | Sorbitan oleate | 1-50 | 1-50 |
| 12. | Flavor | q.s | q.s |
| 13. | Water | q.s | q.s |

Procedure: The composition disclosed in examples 1 and 2 were prepared by adding to purified water, paracetamol, ibuprofen, Magnesium aluminum silicate, Xanthan gum, Liquid maltitol, Sodium benzoate, Saccharin sodium, Polysorbate 80, and Sorbitan oleate, followed by mixing to get a suspension. The pH of the obtained suspension was adjusted between 2-6 by citric acid and suitable flavor was added to it.

### Example 3 and 4

Table 2 provides composition of batches of the present invention.

TABLE 2

| | | Example 3 | Example 4 |
|---|---|---|---|
| SN | Ingredients | mg/5 ml | mg/5 ml |
| 1. | Paracetamol | 250 | 120 |
| 2. | Ibuprofen | 120 | 60 |
| 3. | Magnesium aluminum silicate | 5-150 | 5-150 |
| 4. | Xanthan gum | 0.5-50 | 0.5-50 |
| 5. | Glycerol | 5-250 | 5-250 |
| 6. | Liquid maltitol | 1000-6000 | 1000-6000 |
| 7. | Sodium benzoate | 1-25 | 1-25 |
| 8. | Citric acid | 5-100 | 5-100 |
| 9. | Saccharin sodium | 1-30 | 1-30 |
| 10. | Polysorbate 80 | 1-50 | 1-50 |
| 11. | Sorbitan oleate | 1-50 | 1-50 |
| 12. | Flavor | q.s | q.s |
| 13. | Water | q.s | q.s |

Procedure: The composition disclosed in examples 3 and 4 were prepared by adding to purified water, paracetamol, ibuprofen, Magnesium aluminum silicate, Xanthan gum, Liquid maltitol, Sodium benzoate, Saccharin sodium, Polysorbate 80, and Sorbitan oleate, followed by mixing to get a suspension. The pH of the obtained suspension was adjusted between 2-6 by citric acid and suitable flavor was added to it.

### Example 5 and 6

Table 3 provides composition of batches of the present invention.

TABLE 3

| | | Example 5 | Example 6 |
|---|---|---|---|
| SN | Ingredients | mg/5 ml | mg/5 ml |
| 1. | Paracetamol | 450 | 500 |
| 2. | Ibuprofen | 200 | 80 |
| 3. | Magnesium aluminum silicate | 5-150 | 5-150 |
| 4. | Xanthan gum | 0.5-50 | 0.5-50 |
| 5. | Glycerol | 5-250 | 5-250 |
| 6. | Liquid maltitol | 1000-6000 | 1000-6000 |
| 7. | Sodium benzoate | 1-25 | 1-25 |
| 8. | Citric acid | 5-100 | 5-100 |
| 9. | Saccharin sodium | 1-30 | 1-30 |
| 10. | Polysorbate 80 | 1-50 | 1-50 |
| 11. | Sorbitan oleate | 1-50 | 1-50 |
| 12. | Flavor | q.s | q.s |
| 13. | Water | q.s | q.s |

Procedure: The composition disclosed in examples 5 and 6 were prepared by adding to purified water, paracetamol, ibuprofen, Magnesium aluminum silicate, Xanthan gum, Liquid maltitol, Sodium benzoate, Saccharin sodium, Polysorbate 80, and Sorbitan oleate, followed by mixing to get a suspension. The pH of the obtained suspension was adjusted between 2-6 by citric acid and suitable flavor was added to it.

While the present invention has been described in terms of its specific embodiments, certain modifications and equivalents will be apparent to those skilled in the art and are intended to be included within the scope of the present invention.

We claim:

1. An oral pharmaceutical suspension comprising 100-500 mg/5 ml of paracetamol, 40-80 mg/5 ml of ibuprofen and one or more pharmaceutically acceptable excipients.

2. The oral pharmaceutical suspension of claim 1, wherein the suspension comprises 120 mg/5 ml of paracetamol and 60 mg/5 ml of ibuprofen.

3. The oral pharmaceutical suspension of claim 1, wherein pharmaceutically acceptable excipients comprises one or more of suspending or viscosity increasing agents, sweeteners, buffering agents, preservatives, wetting agents, flavoring agents, solvents.

4. The oral pharmaceutical suspension of claim 3, wherein the suspending or viscosity increasing agents comprise one or more of xanthan gum, guar gum, tragacanth, acacia, gelatin, carrageenan, agar-agar, povidone, alginic acid, sodium alginate, propylene glycol alginate, carbomer, magnesium aluminium silicate, carboxymethylcellulose calcium, sodium carboxymethylcellulose, ethylcellulose, methylcellulose, hydroxypropyl methylcellulose, hydroxyethylcellulose, hydroxypropylcellulose, microcrystalline cellulose, polydextrose, sucrose, sorbitol, xylitol, dextrose, fructose, maltitol, bentonite, polyvinyl alcohol, colloidal silicon dioxide.

5. The oral pharmaceutical suspension of claim 3, wherein the sweeteners comprise one or more of sucrose, sorbitol, xylitol, dextrose, fructose, maltitol, acesulfame potassium, aspartame, saccharin, saccharin sodium, liquid maltitol, liquid glucose, cyclamate, sodium cyclamate.

6. The oral pharmaceutical suspension of claim 3, wherein the buffering agents comprise one or more of citric acid, sodium citrate, sodium phosphate, potassium citrate.

7. The oral pharmaceutical suspension of claim 3, wherein the preservatives comprise one or more of sodium benzoate, benzoic acid, ethylenediaminetetraacetic acid, sorbic acid, bronopol, butyl paraben, methyl paraben, ethylparaben, propyl paraben, sodium propionate, chlorhexidine, potassium sorbate, propylene glycol, sodium bisulfite, sodium metabisulfite, sodium salts of hydroxybenzoate.

8. The oral pharmaceutical suspension of claim 3, wherein the wetting agents comprise one or more of polyethylene glycol, polysorbates, sorbitan esters.

9. The oral pharmaceutical suspension of claim 3, wherein the flavoring agents comprise one or more of artificial strawberry flavor, artificial cream flavor, vanilla, cherry, raspberry.

10. The oral pharmaceutical suspension of claim 3, wherein the solvents comprise one or more of water, glycerol, propylene glycol, polyethylene glycol, ethanol.

11. The oral pharmaceutical suspension of claim 1, wherein the pH of the suspension is in the range of 2 to 6.

12. An oral pharmaceutical suspension comprising 200-450 mg/5 ml of paracetamol, 100-200 mg/5 ml of ibuprofen and one or more pharmaceutically acceptable excipients.

13. The oral pharmaceutical suspension of claim 12, wherein the suspension comprises 250 mg/5 ml of paracetamol and 120 mg/5 ml of ibuprofen.

14. The oral pharmaceutical suspension of claim 12, wherein pharmaceutically acceptable excipients comprises one or more of suspending or viscosity increasing agents, sweeteners, buffering agent, preservatives, wetting agents, flavoring agent, solvents.

15. The oral pharmaceutical suspension of claim 14, wherein the suspending or viscosity increasing agents comprise one or more of xanthan gum, guar gum, tragacanth, acacia, gelatin, carrageenan, agar-agar, povidone, alginic acid, sodium alginate, propylene glycol alginate, carbomer, magnesium aluminium silicate, carboxymethylcellulose calcium, sodium carboxymethylcellulose, ethylcellulose, methylcellulose, hydroxypropyl methylcellulose, hydroxyethylcellulose, hydroxypropylcellulose, microcrystalline cellulose, polydextrose, sucrose, sorbitol, xylitol, dextrose, fructose, maltitol, bentonite, polyvinyl alcohol, colloidal silicon dioxide.

16. The oral pharmaceutical suspension of claim 14, wherein the sweeteners comprise one or more of sucrose, sorbitol, xylitol, dextrose, fructose, maltitol, acesulfame potassium, aspartame, saccharin, saccharin sodium, liquid maltitol, liquid glucose, cyclamate, sodium cyclamate.

17. The oral pharmaceutical suspension of claim 14, wherein the buffering agents comprise one or more of citric acid, sodium citrate, sodium phosphate, potassium citrate.

18. The oral pharmaceutical suspension of claim 14, wherein the preservatives comprise one or more of sodium benzoate, benzoic acid, ethylenediaminetetraacetic acid, sorbic acid, bronopol, butyl paraben, methyl paraben, ethylparaben, propyl paraben, sodium propionate, chlorhexidine, potassium sorbate, propylene glycol, sodium bisulfite, sodium metabisulfite, sodium salts of hydroxybenzoate.

19. The oral pharmaceutical suspension of claim 14, wherein the wetting agents comprise one or more of polyethylene glycol, polysorbates, sorbitan esters.

20. The oral pharmaceutical suspension of claim 14, wherein the flavoring agents comprise one or more of artificial strawberry flavor, artificial cream flavor, vanilla, cherry, raspberry.

21. The oral pharmaceutical suspension of claim 14, wherein the solvents comprise one or more of water, glycerol, propylene glycol, polyethylene glycol, ethanol.

22. The oral pharmaceutical suspension of claim 14, wherein the pH of the suspension is in the range of 2 to 6.

23. A method of treating preoperative, perioperative or postoperative pain by administering to a subject a therapeutically effective amount of oral pharmaceutical suspension comprising 100-500 mg/5 ml of paracetamol and 40-80 mg/5 ml of ibuprofen.

24. The method of claim 23, wherein preoperative, perioperative or postoperative pain is associated with one or more surgeries.

25. The method of claim 24, wherein surgeries comprise one or more of throat, dental, ear or nose surgery.

26. The method of claim 23, wherein the said subject is mammal.

27. A method of treating preoperative, perioperative or postoperative pain by administering to a subject a therapeutically effective amount of oral pharmaceutical suspension comprising 200-450 mg/5 ml of paracetamol and 100-200 mg/5 ml of ibuprofen.

28. The method of claim 27, wherein preoperative, perioperative or postoperative pain is associated with one or more surgeries

29. The method of claim 28, wherein surgeries comprise one or more of throat, dental, ear or nose surgery.

30. The method of claim 27, wherein the said subject is mammal.
